(19) United States
(12) Patent Application Publication
(10) Pub. No.: US 2026/0216508 A1
(43) Pub. Date: Jul. 30, 2026

(54) SYSTEMS AND METHODS OF OPENING THE EUSTACHIAN TUBE

(71) Applicant: GLIMPSE DIAGNOSTICS, INC., Minneapolis, MN (US)

(72) Inventor: Courtney Anne Hill, Arden Hills, MN (US)

(21) Appl. No.: 19/428,054

(22) Filed: Dec. 19, 2025

Related U.S. Application Data
(60) Provisional application No. 63/749,178, filed on Jan. 24, 2025.

(51) Int. Cl. A61N 1/36; A61N 1/04

(52) U.S. Cl. CPC A61N 1/36003; A61N 1/0452; A61N 1/36031; A61N 1/36034

(57) ABSTRACT

A method for opening a Eustachian tube of a patient includes: placing an electrical stimulation device at a patient; applying, via the electrical stimulation device, electrical stimulation to a masseter muscle of a patient according to a stimulation protocol; and as a result of applying the electrical stimulation to the masseter muscle, causing the Eustachian tube of the patient to open.

### RELATED CASE

This disclosure claims priority to US provisional patent application number 63/749,178, filed on Jan. 24, 2025, the entire contents of which are hereby incorporated by reference.

### TECHNICAL FIELD

This disclosure relates to devices, systems, and methods for opening a Eustachian tube of a patient. Disclosed herein are embodiments that apply stimulation to one or more anatomic regions of interest to thereby cause a Eustachian tube of a patient to open. Causing the Eustachian tube to open can result in one or more therapeutic benefits, such as to the middle ear of the patient.

### BACKGROUND

The middle ear space is defined by the tympanic membrane laterally and the Eustachian tube medially. The Eustachian tube connects the middle ear space to the nasopharynx and sits in a closed position at rest. The body's natural mechanism of maintaining equal pressure within the middle ear space relative to the nasopharynx and outside world is by opening the Eustachian tube. It is also the natural mechanism that allows fluid in the middle ear to drain into the nasopharynx. The consequences of Eustachian tube dysfunction can include acute otitis media, otitis media with effusion, tympanic membrane atelectasis, tympanic membrane retraction, cholesteatoma formation, hearing loss, and others.

The Eustachian tube can be opened passively with jaw and throat motions such as chewing or swallowing. It can be opened when there is enough pressure built up in the pharynx to force it open such as when performing a Valsalva maneuver against closed lips and nose. There are products for facilitating this such as a balloon to blow up within the nose instead of the mouth. This is an attempt to allow children to be able to self-insufflate their middle ears by creating the pressure needed to open the Eustachian tube and equalize the pressure. This can be used to help treat Eustachian tube dysfunction. However young children who most often have Eustachian tube dysfunction typically cannot perform this maneuver or suitably receive the available products.

As such, Children are currently treated by surgically placing pressure equalization tubes. This is a tube placed across the tympanic membrane that allows for the equalization of pressure in the middle ear space that is independent of the Eustachian tube. Ear tubes simply equalize the pressure in the middle ear with the pressure in the ear canal (the same as the outside world). This is a surgical procedure, typically requiring anesthesia. It is temporary; tubes typically remain in the tympanic membrane for about 1 year. Once the tubes eject from the tympanic membrane, and the membrane heals the hole where the tube had been sitting, the middle ear must rely on the natural mechanism of the Eustachian tube to equalize pressure and drain fluid. Pressure equalization tubes temporarily fix the situation but do not address the underlying problem of Eustachian tube dysfunction.

### SUMMARY

This disclosure is directed to devices, systems, and methods for opening a Eustachian tube of a patient. Such embodiments can cause the Eustachian tube of the patient to open by applying stimulation, such as electrical energy stimulation, to one or more anatomic regions of interest of the patient to thereby cause one or both Eustachian tubes of that patient to open. Causing the Eustachian tube to open can result in one or more therapeutic benefits to the patient. For instance, causing the Eustachian tube to open can help to equalize pressure within the middle ear and/or help to drain fluid (infected or non-infected; e.g., mucus) from the middle ear. By applying electrical stimulation to a patient to actuate opening of the Eustachian tube, embodiments disclosed herein can leverage and induce the body's natural mechanism for equalizing pressure at and draining fluid from the middle ear. Such embodiments can be particularly useful in application on children, who tend to be unable or unsuited for inducing Eustachian tube opening with balloons or Valsalva maneuvers and who may be wary and unaccommodating to physical impact stimulation (e.g., hitting the child's jaw).

One embodiment includes a method for opening a Eustachian tube of a patient. This method embodiment includes the steps of: placing an electrical stimulation device at a patient; applying, via the electrical stimulation device, electrical stimulation to a masseter muscle of a patient according to a stimulation protocol; and as a result of applying the electrical stimulation to the masseter muscle, causing the Eustachian tube of the patient to open.

In a further embodiment, the method can additionally include receiving feedback from the patient to determine whether the Eustachian tube of the patient is open or has opened. In some such examples, the method can further include, when the feedback from the patient indicates that the Eustachian tube of the patient is open or has opened, terminating applying, via the electrical stimulation device, electrical stimulation to the masseter muscle of the patient. In certain such examples, the method can further include the steps of: after terminating applying, via the electrical stimulation device, the electrical stimulation to the masseter muscle of the patient, again applying, via the electrical stimulation device, electrical stimulation to the masseter muscle of the patient; and, as a result of again applying electrical stimulation to the masseter muscle of the patient, causing the Eustachian tube of the patient to open a second time.

In some such embodiments, the stimulation protocol can be a first stimulation protocol. The method can additionally include, when the feedback from the patient indicates that the Eustachian tube of the patient is not open or did not open over a predetermined period of time (e.g., the Eustachian tube did not open over a given day), applying, via the electrical stimulation device, electrical stimulation to the masseter muscle of the patient according to a second, different stimulation protocol. For instance, the second, different stimulation protocol can include a greater electrical energy intensity than the first stimulation protocol and/or a longer duration application of electrical energy than the first stimulation protocol. The first stimulation protocol an include an electrical energy intensity ranging from 1 to 30 mA and a duration of electrical energy application ranging from 0.1 to 0.5 milliseconds.

In some such embodiments, the method can additionally include using the feedback received from the patient to determine whether to apply the electrical stimulation to the masseter muscle of the patient unilaterally or bilaterally. In some embodiments, the feedback from the patient can be received via sonotubometry and/or the feedback from the patient can include other middle ear measurements or middle ear imaging that indicate fluid remains at the middle ear and/or a metric corresponding to compliance of a tympanic membrane of the patient.

In some such embodiments, applying the electrical stimulation to the masseter muscle to cause the Eustachian tube of the patient to open can include applying electrical energy to stimulate a masseter reflex associated with the masseter muscle to cause contraction of a tensor veli palatini of the patient to thereby open the Eustachian tube of the patient.

In some such embodiments, the method can additionally include, prior to applying, via the electrical stimulation device, electrical stimulation to the masseter muscle of the patient, acquiring middle ear status information corresponding to an ear having the Eustachian tube of the patient. For example, the acquired middle ear status information can include a fluid status associated with the middle ear and/or a pressure status associated with the middle ear. The electrical stimulation can be applied to the masseter muscle of the patient via the electrical stimulation device when the acquired middle ear status information indicates a presence of fluid at the middle ear of the patient exceeding a predetermined fluid threshold and/or when the acquired middle ear status information indicates pressure at the middle ear of the patient is abnormal, for instance, because it deviates from a predetermined pressure range (e.g., a predetermined pressure range associated with normal, expected middle ear pressure). In some applications, the predetermined fluid threshold can include both a volume of fluid present predetermined threshold and a fluid viscosity predetermined threshold. It is noted that the predetermined pressure threshold can relate to positive and/or negative pressure measurements and can provide a predetermined threshold (positive or negative) to which to compare middle ear pressure measurement(s) to in order to determine whether such middle ear pressure measurement(s) are abnormal. As such, in some examples an absolute value of the pressure measurement(s) relating to the middle ear can be compared to the predetermined pressure threshold to determine whether the detected pressure at the middle ear is abnormal. As noted, in some applications, the predetermined pressure threshold can include a negative pressure at the middle ear.

In some such embodiments, the electrical stimulation device can be placed at an external skin surface of the patient, and the electrical stimulation device can include a surface electrode.

In some such embodiments, placing the electrical stimulation device at the patient can include placing the electrical stimulation device directly at the masseter muscle of the patient such that electrical energy associated with the electrical stimulation is directly applied to the masseter muscle. In other such embodiments, placing the electrical stimulation device at the patient can include placing the electrical stimulation device indirectly at the masseter muscle of the patient such that electrical energy associated with the electrical stimulation is indirectly applied to the masseter muscle through at least one other anatomic portion of the patient.

Another embodiment disclosed herein is a closed-loop system for acquiring ear status information of a patient and applying stimulation (e.g., electrical stimulation energy) to that patient.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present invention and therefore do not limit the scope of the invention. The drawings are not necessarily to scale, though embodiments can include the scale illustrated, and are intended for use in conjunction with the explanations in the following detailed description wherein like reference characters denote like elements. Examples of the present invention will hereinafter be described in conjunction with the appended drawings.

FIG. 1 is a block diagram of an embodiment of a closed loop system for acquiring ear status information and applying stimulation (e.g., electrical stimulation energy) to a patient.

FIG. 2 is a flow diagram of an embodiment of a method for using middle ear status information to determine whether to apply stimulation (e.g., and according to a preset stimulation protocol) to a patient. For example, the flow diagram shown at FIG. 2 can represent decision points and possible responses within the system of FIG. 1 based on the inputs received.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

The Eustachian tube opening is controlled by the levator palatini and tensor palatini muscles. There is evidence to suggest that the tensor palatini muscle is more influential and stimulation of this muscle alone causes the tube to open. The tensor palatini is controlled by the trigeminal nerve (cranial nerve V). The mandibular branch of the trigeminal nerve carries both sensory and motor fibers to the bottom third of the face. The motor fibers supply the tensor palatini muscle. The mandibular nerve courses through the mandible and exits as the mental nerve at the mental foramen, typically located between the roots of the first and second premolar teeth.

By stimulating the branches of the mandibular nerve with afferent or efferent travel depending on the site of the stimulation, the motor fibers to the tensor veli palatini muscle can be stimulated, leading to opening of the Eustachian tube, equalization of pressure within the middle ear, drainage of built up fluid (infected or non-infected) and mucus. This would be a beneficial event for humans suffering from Eustachian tube dysfunction or its consequences by inducing and relying on the body's natural mechanism for equalizing middle ear pressures.

There are multiple ways of stimulating the trigeminal nerve directly or indirectly. Branches of this nerve surfacing below the mucosa in the mouth provides a target for electrical stimulation, either transmucosally inside the mouth or transcutaneously on the chin. This would stimulate the mandibular branch of the trigeminal nerve.

Another target of electrical stimulation can be the auriculotemporal nerve, a branch of the mandibular nerve, ultimately part of the trigeminal nerve. This could be effectively stimulated transcutaneously either with a needle inserted or with surface electrodes in the temple region. Another target of electrical stimulation can be the lingual branch of the marginal nerve. Transmucosal stimulation could occur anywhere along the course of the lingual branch, including under the tongue.

Indirect stimulation of nerves can occur when reflex pathways exist. Another method of stimulating the trigeminal nerve that ultimately results in motor output from the trigeminal nerve includes taking advantage of an existing reflex pathway. The jaw jerk or masseter reflex is one such pathway. Stimulating this reflex can specifically cause contraction of the tensor veli palatini. The masseter reflex takes in sensory information from the mandibular branch of the trigeminal nerve and travels via the mesencephalic tract to the mesencephalic nucleus of the trigeminal nerve in the brainstem. It communicates with the trigeminal motor nucleus and the output is a motor signal to contract the muscles of the first branchial arch, which are innervated by the trigeminal nerve. These include the tensor veli palatini, in addition to the masseter, temporalis, pterygoids, mylohyoid, and anterior belly of digastric muscles. Stimulating the masseter reflex therefore causes contraction of the tensor veli palatini and opens the Eustachian tube.

Normally the masseter reflex is observed by tapping the jaw with the mouth open and relaxed. When the jaw is open, it puts the masseter muscle on stretch, which in turn allows the muscle spindles to be stimulated with the tap on the jaw. Masseter muscle spindle stimulation is what triggers this reflex to take place and ultimately it causes the jaws to close. Masseter muscle spindle fibers are activated when the muscle is under stretch, such as when a bite block is placed to open the mouth. The masseter reflex can be induced in adults with electrical stimulation of the masseter muscle.

Electrical stimulation for muscles can encompass a wide range of methods and protocols designed to target various physiological outcomes and within the scope of the present disclosure. Types of stimulation include Neuromuscular Electrical Stimulation (NMES) for eliciting muscle contractions, Functional Electrical Stimulation (FES) to facilitate functional movements, Transcutaneous Electrical Nerve Stimulation (TENS) for pain relief, Interferential Current (IFC) using overlapping currents, and high-frequency Russian Stimulation for strength training. Protocols vary with waveform shapes (monophasic, biphasic, polyphasic) and pulse configurations (square, sine, triangular), adjusted across frequencies (low, medium, high) and intensities (sub-threshold to suprathreshold). Applications can utilize electrode placements (bipolar, monopolar, quadripolar), custom duty cycles, ramp times, and session durations, ranging from short intermittent bursts to continuous stimulation. Advanced methods can integrate multimodal therapies (e.g., ultrasound or vibration).

Electrical stimulation can be transcutaneous, transmucosal, percutaneous, permucosal. Ultrasound or other imaging modalities might be used in conjunction to guide electrical stimulation needle or probe when any of these transcutaneous, transmucosal, percutaneous, permucosal modalities are used.

In the application for stimulating the trigeminal nerve, it could be unilateral or bilateral. If unilateral stimulation is performed, it can be done alone or sequentially with the opposite side. A stimulation protocol can include alternating between sides, with the length of time spent on each varying based on the physiologic response. Stimulation of the trigeminal nerve could be performed at one target or multiple targets along the branches of the mandibular nerve. One or more targets of stimulation can be stimulated simultaneously, sequentially, or in a pattern found to be most beneficial. The depth and point of stimulation could be in direct contact with the nerve or anywhere between the surface of the skin and the nerve itself.

A device and/or system within the scope of the present disclosure could include a module that identifies the underlying anatomy with ultrasound or other imaging, guides a needle or probe to the correct depth, sends electrical signals with any of the stimulation protocols as noted above, and, in some applications, can modulate the electrical signals based on feedback from the patient.

Electrical stimulation of the masseter spindle fibers can be done with any one or more of a variety of electrical stimulation devices. For example, suitable electrical stimulation devices can apply electrical stimulation to the masseter muscle directly with a needle electrode inserted into the muscle, indirectly with a needle electrode inserted anywhere between the muscle and the skin, or directly or indirectly with surface electrodes on the skin. A stimulation protocol could range from 0.1 millisecond to 1.0 millisecond (e.g., ranging from 0.1 ms to 0.5 ms, such as 0.2 ms duration), 0.7 Hz repetition, and electrical energy intensity ranging 1 mA to 30 mA.

Nerve stimulation can also be achieved with acupressure or acupuncture to certain anatomical points or broadly to stimulate the underlying nerves.

Opening the Eustachian tube causes equalization of pressure in the middle ear space with the nasopharynx pressure (i.e. the outside world pressure). Eustachian tube dysfunction is when the Eustachian tubes do not naturally open and equalize the pressure when there is a negative pressure in the middle ear. This failure is the underlying cause of otitis media, including acute otitis media and otitis media with effusion, in young children. With chronic Eustachian tube dysfunction, this typically leads to abnormalities of the middle ear including atelectasis of the tympanic membrane, cholesteatoma formation which can lead to erosion of the middle ear bones or erosion of the temporal bone leading to an abnormal connection between the middle ear and the intracranial space. Erosion of the middle ear bones leads to hearing loss.

Detection of the Eustachian tube opening is important for a system that induces the Eustachian tube to open. It can serve as confirmation of success and also an endpoint of electrical stimulation. A feedback loop of confirming that the Eustachian tube has opened or has remained closed allows for a decision analysis for a computer system to continue to stimulate the tensor veli palatini (e.g., the masseter muscle), either indirectly or directly. For instance, if the feedback from the patient indicates that the Eustachian tube of the patient is open or has opened, the electrical stimulation applied via the electrical stimulation device to the masseter muscle of the patient can be terminated. Or, if the feedback from the patient indicates that the Eustachian tube of the patient is not open or did not open, the stimulation protocol can be changed so as to apply electrical stimulation via the electrical stimulation device to the masseter muscle of the patient according to a different electrical stimulation protocol. One method of detecting whether the Eustachian tube has opened is sonotubometry. Sonotubometry is a continuous measurement of transmission of sound from the nose to the ear canal. This involves a sound producing probe being placed in the nostril and a microphone probe placed in the ear canal that records sound pressure. When the Eustachian tube opens, an increase in sound will be recorded in the ear canal; the sound travels from the nose, through the Eustachian tube across the eardrum, and into the ear canal. Another method of detecting whether the Eustachian tube has opened is by measuring the compliance of the tympanic membrane through tympanometry. This is an indirect measure of the middle ear pressure. If the middle ear pressure has equalized to the outside world, ie the Eustachian tube has opened, the tympanometry tracing will be a type A. If there is negative pressure, the tympanometry tracing will be a type C. If there is fluid in the middle ear (infected or not infected, and any viscosity), the tympanogram will be a type B.

By opening the Eustachian tube, this could allow for any fluid to drain into the nasopharynx. This fluid could be infected fluid such as in acute middle ear infection or non-inflected fluid that ranges from thin to thick, such as serous, mucoid, seromucoid, mucopurulent, or “glue ear.” Repeated opening of the Eustachian tube might be more effective at draining the thicker fluid.

Tympanometry can be used to confirm the middle ear diagnosis by the healthcare provider. This can be a pre-treatment measurement. Tympanometry can be used to confirm that the Eustachian tube opened and equalized the pressure during treatment. Tympanometry can be used to confirm that fluid has drained from the middle ear during treatment. Tympanometry can be used in real time during treatment to assess at specific intervals whether the treatment has successfully caused the Eustachian tube to open, to equalize pressure in the middle ear, or to drain the middle ear fluid that existed pre-treatment.

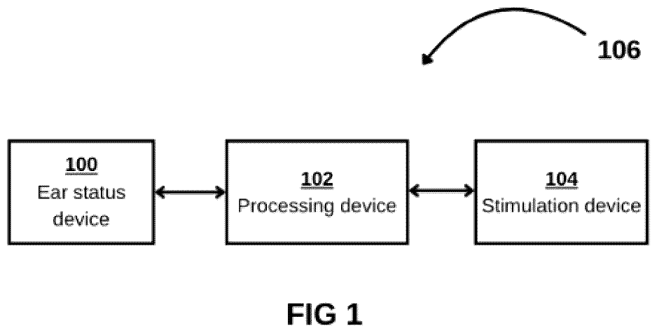

FIG. 1 illustrates a block diagram of an embodiment of a closed loop system 106 for acquiring ear status information and applying stimulation (e.g., electrical stimulation energy) to a patient. The closed loop system 106 includes an ear status assessment device 100, processing device 102, and stimulation device 104 (e.g., electrical stimulation device). The ear status assessment device 100 can be configured to assess one or more statuses associated with an ear, such as one or more statuses related to the middle ear (e.g., fluid and/or pressure status associated with the middle ear). The processing device 102 can receive data from the ear status assessment device 100, such as the data relating to one or more statuses associated with the ear, and the processing device 102 can execute non-transitory computer readable instructions to at least compare the data received from the ear status assessment device 104 to one or more predetermined ear status thresholds. For example, the processing device 104 can compare middle ear fluid and/or pressure related data from the ear status assessment device 100 to one or more predetermined ear fluid thresholds and/or to one or more predetermined ear pressure thresholds. Using the data from the ear status assessment device 100 (e.g., based on the one or more comparisons), the processing device 102 can cause the stimulation device 104 to apply stimulation to a region of interest (e.g., the Masseter muscle) at the patient. The stimulation device 104 can be placed at the patient and configured to apply stimulation energy, such as electrical energy, to the region of interest according to one or more parameters of a stimulation protocol.

While, and/or after, the stimulation is applied to the region of interest, the ear status assessment device 100 can procure additional data pertaining to one or more statuses of the ear (e.g., middle ear), and the processing device 104 can receive this additional data and execute the non-transitory computer readable instructions to at least compare the additional data received from the ear status assessment device 100 to one or more predetermined ear status thresholds, and the processing device can use this comparison to determine whether to alter the stimulation being applied, or that was previously applied, to the patient. In this way, the system 106 can provide a closed-loop feedback system for ear therapeutic stimulation applications that can iteratively (e.g., in substantially real-time) diagnose the status of the ear (e.g., middle ear) and use this diagnosed status of the middle ear to initiate a stimulation protocol and/or subsequently alter this stimulation protocol to tailor the stimulation protocol to the diagnostic information received from the ear status assessment device 100, for instance, by adjusting one or more parameters of the stimulation protocol based on the substantially-real time diagnosed status of the middle ear.

In some examples, a non-transitory storage medium can store two or more tailored stimulation protocols, and the processing device 102 can be configured to select a tailored stimulation protocol corresponding to application of stimulation to the patient for whom the ear status diagnostic information corresponds. Thus, the processing device 102 can change the initial stimulation protocol based on real-time patient diagnostic information by selecting from the two or more tailored stored stimulation protocols that most closely corresponds to the real-time patient diagnostic information.

Described is a system and methods to treat Eustachian tube dysfunction, negative pressure in the middle ear, middle ear fluid, middle ear infection, or any combination of these diagnoses. The closed loop system 100 includes a device 102 for assessing one or more status of the ear (e.g., for assessing two or more statuses of the middle ear); a processing module 104; and a stimulation device 106. The device 102 for assessing middle ear status can be a probe in the ear for tympanometry measurements, a probe in the ear for sonotubometry measurements (as well as a probe in the nose for sound production), a probe in the ear to capture images of the tympanic membrane. It can also be a probe in the ear to deliver pneumatic otoscopy, traditional otoscopy, or video otoscopy. It can also be a probe capable of performing Acoustic Reflectometry, Wideband Tympanometry, Otoacoustic Emissions, Laser Doppler Vibrometry, Auditory Brainstem Response, Impedance Audiometry, Wideband Absorbance Immittance, Optical Coherence Tomography, Spectral Gradient Acoustic Reflectometry, Ultrasound or any combination of these. The point is to detect and characterize the contents of the middle ear, which can include detection of the viscosity of the fluid present. The information from the probe is sent to the computing module which interprets the middle ear status based on the inputs. The interpretation can include an assessment of a tympanometry tracing, an assessment of an image containing the tympanic membrane, an assessment of sonotubometry tracing, as examples, or any other data from the entities noted above which can be interpreted to understand the characteristics of the middle ear contents. The interpretation can be carried out by software that employs algorithms, computer vision assessments, machine learning, deep learning, or a combination of methods. The interpretation of the middle ear status can determine characteristics of the middle ear such as whether the pressure is equalized or whether there is fluid present (infected or non infected), with consideration of the viscosity if fluid is present.

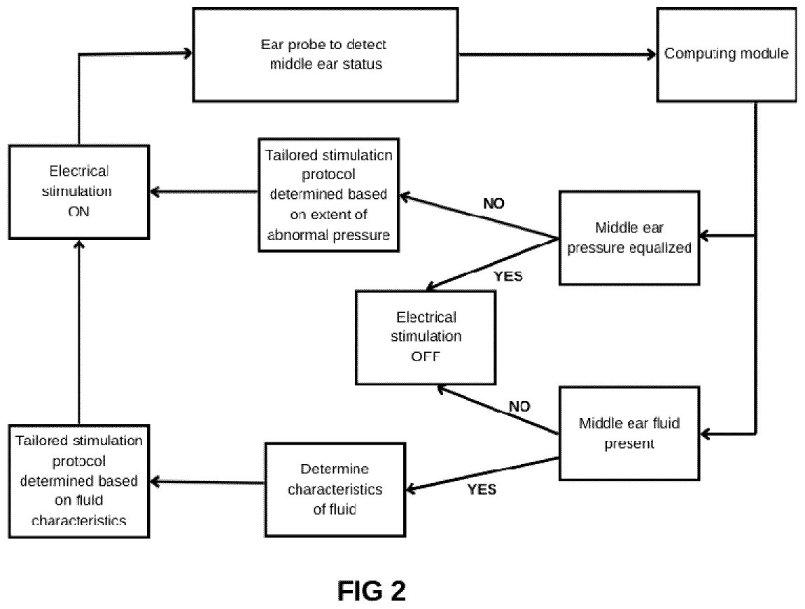

FIG. 2 illustrates a flow diagram of an embodiment of a method for opening a Eustachian tube of a patient using electrical stimulation. The example at FIG. 2 shows decision points and possible responses, such as executed within the system 106 of FIG. 1 based on the feedback/inputs received. If the middle ear pressure is not equalized, then the computing module can direct the electrical stimulation module to stimulate the tensor palatini, directly or indirectly through methods described above. The status of the middle ear is assessed at the end of the electrical stimulation and information is sent to the computing module for interpretation. If the middle ear pressure has equalized, the electrical stimulation module will be turned off and the treatment complete. If the interpretation of the middle ear status demonstrates that there is middle ear fluid present, the computing module can direct the electrical stimulation module to stimulate tensor palatini directly or indirectly. At the end of the electrical stimulation, the middle ear status is assessed and information sent from the probes to the computing module for interpretation. If the computing module assesses that the middle ear fluid is no longer present, then the computing module can direct the electrical stimulation to turn off and the treatment complete.

If repeated assessments of the middle ear status demonstrate persistent middle ear fluid or middle ear pressure continues to be negative instead of equalized, the electrical stimulation module can be modulated by the computing module to change the electrical stimulation protocol from baseline. In this way, the stimulation protocol is tailored to the input of ear status. This could change throughout treatment, in response to each time the ear status is checked. This could involve increasing or decreasing intensity, lengthening or shortening stimulation time, increasing or decreasing frequency. A maximum of intensity, stimulation time, and frequency can be set by the user or preset in the system.

The ear status assessment by the ear probe can gather information about the characteristics, such as viscosity, of the fluid present and the computing module can have logic that modulates the electrical stimulation protocol based on these characteristics. For example, if there is a very viscous middle ear fluid, perhaps higher intensity and longer stimulation times might be needed to enact longer muscle contractions of the tensor veli palatini to maximize the opening time of the Eustachian tube. It can also be used to determine whether unilateral or bilateral stimulation should occur. The ear status assessment can happen simultaneously or sequentially in both ears.

Special attention can be given to interpretations by the computing module of persistent ear fluid in spite of electrical stimulation. The diameter of the opening of the Eustachian tube can influence the amount and type of fluid that can pass through. It is possible that a wider diameter opening is needed to drain the ear of more viscous fluid or infection. The degree of opening or width of Eustachian tube opening can be detected by feedback in the ear canal, including but not limited to the level of sound. This can be measured with a probe in the ear and can serve as additional information to the computing module to assess the extent to which the Eustachian tube opens. The computing module can direct the electrical stimulation module to modulate the stimulation parameters of intensity, frequency, and stimulation time based on the extent to which the Eustachian tube opens, thus allowing a tailored stimulation.

This system can be used in the setting of indirect stimulation of the tensor palatini muscle to open the Eustachian tube, such as through invoking the masseter reflex. In that case, the system also includes a bite block or other material that maintains the jaws in an open position in order to stretch the masseter spindle fibers. In humans who are edentulous, such as young children, the bite block will need to be adapted. For example, a soft material that can mold to the jaw of the user to act as a cushion between the gums of the jaws would be useful. Additional consideration for children who are partially dentulous could combine the characteristics of a traditional bite block and the described molded cushion.

What is claimed is:

1. A method for opening a Eustachian tube of a patient, the method comprising the steps of:
placing an electrical stimulation device at a patient;
applying, via the electrical stimulation device, electrical stimulation to a masseter muscle of a patient according to a stimulation protocol; and
as a result of applying the electrical stimulation to the masseter muscle, causing the Eustachian tube of the patient to open.

2. The method of claim 1, further comprising:
receiving feedback from the patient to determine whether the Eustachian tube of the patient is open or has opened.

3. The method of claim 2, further comprising:
when the feedback from the patient indicates that the Eustachian tube of the patient is open or has opened, terminating applying, via the electrical stimulation device, electrical stimulation to the masseter muscle of the patient.

4. The method of claim 3, further comprising:
after terminating applying, via the electrical stimulation device, the electrical stimulation to the masseter muscle of the patient, again applying, via the electrical stimulation device, electrical stimulation to the masseter muscle of the patient; and
as a result of again applying electrical stimulation to the masseter muscle of the patient, causing the Eustachian tube of the patient to open a second time.

5. The method of claim 2, wherein the stimulation protocol is a first stimulation protocol, and further comprising:
when the feedback from the patient indicates that the Eustachian tube of the patient is not open or did not open, applying, via the electrical stimulation device, electrical stimulation to the masseter muscle of the patient according to a second, different stimulation protocol.

6. The method of claim 5, wherein the second, different stimulation protocol comprises a greater electrical energy intensity than the first stimulation protocol and/or a longer duration application of electrical energy than the first stimulation protocol.

7. The method of claim 6, wherein the first stimulation protocol comprises an electrical energy intensity ranging from 1 to 30 mA and a duration of electrical energy application ranging from 0.1 to 0.5 milliseconds.

8. The method of claim 2, further comprising:
using the feedback received from the patient to determine whether to apply the electrical stimulation to the masseter muscle of the patient unilaterally or bilaterally.

9. The method of claim 2, wherein the feedback from the patient is received via sonotubometry.

10. The method of claim 2, wherein the feedback from the patient comprises a metric corresponding to compliance of a tympanic membrane of the patient.

11. The method of claim 1, wherein applying the electrical stimulation to the masseter muscle to cause the Eustachian tube of the patient to open comprises applying electrical energy to stimulate a masseter reflex associated with the masseter muscle to cause contraction of a tensor veli palatini of the patient to thereby open the Eustachian tube of the patient.

12. The method of claim 1, further comprising:
prior to applying, via the electrical stimulation device, electrical stimulation to the masseter muscle of the patient, acquiring middle ear status information corresponding to an ear having the Eustachian tube of the patient.

13. The method of claim 12, wherein the acquired middle ear status information comprises a fluid status associated with the middle ear and/or a pressure status associated with the middle ear.

14. The method of claim 13, wherein the electrical stimulation is applied to the masseter muscle of the patient via the electrical stimulation device when the acquired middle ear status information indicates a presence of fluid at the middle ear of the patient exceeding a predetermined fluid threshold and/or when the acquired middle ear status information indicates a deviation from a predetermined pressure range.

15. The method of claim 14, wherein the predetermined fluid threshold comprises both a volume of fluid present predetermined threshold and a fluid viscosity predetermined threshold.

16. The method of claim 14, wherein the predetermined pressure threshold comprises a negative pressure at the middle ear.

17. The method of claim 1, wherein the electrical stimulation device is placed at an external skin surface of the patient.

18. The method of claim 17, wherein the electrical stimulation device comprises a surface electrode.

19. The method of claim 1, wherein placing the electrical stimulation device at the patient comprises placing the electrical stimulation device directly at the masseter muscle of the patient such that electrical energy associated with the electrical stimulation is directly applied to the masseter muscle.

20. The method of claim 1, wherein placing the electrical stimulation device at the patient comprises placing the electrical stimulation device indirectly at the masseter muscle of the patient such that electrical energy associated with the electrical stimulation is indirectly applied to the masseter muscle through at least one other anatomic portion of the patient.
